(19) United States
(12) Patent Application Publication
(10) Pub. No.: US 2015/0039336 A1
(43) Pub. Date: Feb. 5, 2015

(54) METHOD, PLANNING UNIT AND SYSTEM TO PLAN A MEDICAL EXAMINATION

(71) Applicant: SIEMENS AKTIENGESELLSCHAFT, Muenchen (DE)

(72) Inventors: Klaus Mayer, Eckental (DE); Mike Mueller, Moehrendorf (DE)

(73) Assignee: SIEMENS AKTIENGESELLSCHAFT, Muenchen (DE)

(21) Appl. No.: 14/451,773

(22) Filed: Aug. 5, 2014

(30) Foreign Application Priority Data
Aug. 5, 2013 (DE) 102013215345.7

(51) Int. Cl. G06Q 50/22; A61B 6/03; G06Q 10/06; A61B 5/00; G06F 17/30; A61B 5/055; A61B 8/14

(52) U.S. Cl. CPC G06Q 50/22; A61B 5/055; A61B 6/037; A61B 6/032; A61B 8/14; A61B 5/0059; G06F 17/3028; G06Q 10/06315
USPC 705/2

(57) ABSTRACT

A method to plan at least one examination of an examination subject with at least one medical imaging apparatus by means of a planning unit, includes the following steps. The planning unit is connected with the at least one medical imaging apparatus. At least one input parameter of a database of the planning unit and at least one input parameter of a database of a subset of the at least one medical imaging apparatus are detected. The database of the planning unit and the database of the subset of the at least one medical imaging apparatus are filtered depending on the at least one detected input parameter. At least one item of output information is determined that includes a selection from medical imaging apparatuses available for the examination of the examination subject.

### BACKGROUND OF THE INVENTION

1. Field of the Invention

The present invention concerns: a method to plan at least one medical examination of an examination subject with at least one medical imaging apparatus using a planning unit, as well as a corresponding planning unit, a medical planning system, and a non-transitory, computer-readable storage medium encoded with programming instructions to implement such a method.

2. Description of the Prior Art

The planning of examinations of an examination subject with a medical imaging apparatus is a widespread field of activity, in particular in clinical application.

In the everyday clinical environment, the planning of examinations can be an extremely complex problem, particularly if a number of examination subjects are to be examined with a number of medical imaging apparatuses.

### SUMMARY OF THE INVENTION

An object of the invention is to provide a method that facilitates the planning of such examinations, and with which an association of a medical imaging apparatus with an examination subject is possible depending on various boundary conditions.

In accordance with the invention, a method is provided for planning at least one examination of an examination subject with at least one medical imaging apparatus using a planning unit that includes the following steps:

connect the planning unit with the at least one medical imaging apparatus,

detect at least one input parameter of a database of the planning unit and at least one input parameter of a database of a subset of at least one medical imaging apparatus,

filter the database of the planning unit and the database of the subset of the at least one medical imaging apparatus depending on at least one detected input parameter, and

determine at least one item of output information that includes a selection from medical imaging apparatuses available for the examination of the examination subject.

A medical imaging apparatus is an apparatus, such as an electronic and/or information technology apparatus, to acquire, process, evaluate and/or store image information in the form of image data. For example, acoustic methods such as ultrasound (US); emission methods such as emission computer tomography (ECT) and positron emission tomography (PET); optical methods; radiological methods such as x-ray tomography and computer tomography (CT); magnetic resonance (MR) tomography (MRT), or combined methods can be used to acquire the image information. The medical imaging apparatus can deliver 2-dimensional (2D) or multidimensional image data such as 3-dimensional (3D) or 4-dimensional (4F) image data, which image data can advantageously be stored and/or processed in different formats. The medical imaging apparatus can be used in diagnostics, for example in medical diagnostics.

A planning unit is a system that has a processing unit, a memory unit, an operating system and advantageously an output unit.

A database is a system for electronic data administration, in particular for storing, overwriting and deleting data and for optimizing queries to the database. The database offers a database language for such queries. One input parameter of a database can include a special data set; however, it can also include only one line, one column or even only a single entry of a table. Various input parameters can differ in different levels of scalability; for example, it can be possible to scale them in a nominal, ordinal or cardinal manner.

The connection of the planning unit with the at least one medical imaging apparatus serves for a data exchange between planning unit and the at least one medical imaging apparatus and takes place via a network, for example internet or intranet; an authentication advantageously takes place via a user recognition as well as a password. The planning unit can thereby be connected wirelessly or via wires with the medical imaging apparatus. The connection can also take place with the assistance of a framework such as, for instance, Active Server Pages.NET (ASP.NET).

The detection of at least one input parameter of a database of the planning unit and at least one input parameter of a database of a subset of at least one medical imaging apparatus takes place (for example) manually via a user interface (for instance an input unit of the planning unit) or automatically using a pre-established routine. “Detection” means both the entry of an input parameter via a user interface (for instance of an input unit of the planning unit) and the selection of an input parameter by means of a selection apparatus of the planning unit.

The filtering of the database of the planning unit and of the database of the subset of the at least one medical imaging apparatus advantageously takes place automatically depending on at least one detected input parameter. For example, a detected input parameter can represent a combination of different query criteria, and thus further limit the query given an AND-linking of the query criteria or expand them accordingly given an OR-linking.

The determination of at least one item of output information, which includes a selection from available medical imaging apparatuses for the examination of the examination subject, takes place depending on the filtered database, i.e. using the result of the query that was previously made, i.e. thus using the previously detected input parameters. The output information includes a subset of the set of medical imaging apparatuses that are available in total for an examination of the examination subject. A selection of a defined, preferred medical imaging apparatus can take place manually via a user interface (for instance an input unit of the planning unit) or automatically using a pre-established routine.

The invention uses the direct access of the planning unit to the at least one medical imaging apparatus in order to determine a medical imaging apparatus that is suitable for the examination of the examination subject via a detection of input parameters of the database of the planning unit and the database of a subset of the at least one medical imaging apparatus. The data inventory of a number of medical imaging apparatuses can be queried directly without prior adjustment. This additionally leads to a time savings, both for users of the planning unit and for users of the at least one medical imaging apparatus.

In a preferred embodiment, the at least one detected input parameter of the database of the planning unit, and/or of the at least one detected input parameter of the database of the subset of the at least one medical imaging apparatus, and/or the selection from the available medical imaging apparatuses, are stored in the database of the planning unit and/or in the database of the subset of the at least one medical imaging apparatus. The detection of the input parameters and/or the result of the filtering of the databases can thus be saved. This leads to an additional time savings for queries (in particular future queries) with the same input parameters.

In an embodiment, the database of the planning unit and the database of the subset of the at least one medical imaging apparatus are synchronized after connecting the planning unit with the at least one medical imaging apparatus. As used herein, a “synchronization” is the establishment of the identical data inventory at the planning unit and at the at least one medical apparatus. This serves on the one hand to secure data, on the other hand to shorten the response times to the queries to the databases, in particular for reading data accesses. In addition, it enables a better load distribution of computing processes.

In a further embodiment, the at least one input parameter of the database of the planning unit, and/or the at least one input parameter of the database of the subset of the at least one medical imaging apparatus, is provided by the planning unit and/or by the at least one medical imaging apparatus. In addition to an expansion of the number of available input parameters, this enables a location independency of the input of the input parameters, thus saves additional time and increases the efficiency of an examination planning.

In an embodiment according to the invention, the at least one input parameter of the database of the planning unit includes at least one parameter for identification of the examination apparatus (for instance an individual patient identification number (patient ID)) and/or at least one parameter for identification of the examination (for instance an individual examination identification number (examination ID)) or an identification number for an individual planning order (order ID). This serves for an efficient overview of a specific examination, and facilitates the overview of all planned examinations.

In a preferred embodiment, the at least one input parameter of the database of the subset of the at least one medical imaging apparatus includes at least one measurement protocol, for example a specific examination workflow. It also includes at least one parameter of a measurement protocol, for example a boundary condition of the specific examination workflow. In addition, it also includes at least one parameter of a hardware revision (for example a specific model of the medical imaging apparatus) and/or at least one parameter of a software revision (for example the revision number of an operating software of the medical imaging apparatus). For example, examination workflows are a cardiac MRT, a paranasal sinus CT, etc.; boundary conditions of the specific examination workflow are, for example, an examination duration, a radiation intensity, a magnetic field strength etc. This type of input parameter allows a specific filtering of the databases with regard to suitable medical imaging apparatuses, meaning that only those medical imaging apparatuses that are also considered for the concrete examination workflows, and satisfy all boundary conditions of the specific examination workflows as well as possible requirements for hardware and software of the medical imaging apparatuses, can be designated for an examination.

In another embodiment, at least one input parameter of the database of the planning unit and/or at least one input parameter of the database of the subset of the at least one medical imaging apparatus include an operator information. Operator information is, among other things (but not exclusively), information that is relevant to the examination, be it an instruction to the examination personnel, to an operator of the medical imaging apparatus or to the maintenance personnel of the medical imaging apparatus, or be it also merely information about a medical imaging apparatus. The operator information can include a continuous text, additional parameters or other information that is relevant to the examination of the examination subject. Efficient examinations adapted to the operator information are thereby possible. The supplementation of information is also advantageous when the person who plans the examination does not belong to the set of persons who are participating in the actual examination.

In a further embodiment, a parameter for identification of the examination is additionally associated with said examination and stored in the database of the planning unit and/or in the database of the subset of the at least one medical imaging apparatus. Parameters for identification of the examination are, for example, individual examination identification numbers (examination IDs) or identification numbers for an individual planning job (job IDs). This serves for a better reproducibility of the input parameters belonging to the planned examination and the medical imaging apparatus determined for this purpose.

In an embodiment according to the invention, after storing the at least one detected input parameter the database of the planning unit and/or of the at least one detected input parameter of the database of the subset of the at least one medical imaging apparatus and/or the selection from the available medical imaging apparatuses in the database of the planning unit and/or in the database of the subset of the at least one medical imaging apparatus, the stored data are confirmed by means of the at least one medical imaging apparatus. This serves to avoid errors as they can arise in the rescheduling of an examination. For example, it is thus prevented that an examination cannot be implemented after changing a defined medical imaging apparatus since a measurement protocol or a parameter of a measurement protocol is not available at the changed medical imaging apparatus. By confirming the stored data by means of the changed medical imaging apparatus, the stored data there can be monitored again by a user and checked for consistency. The conformation can thereby inherently take place with, among other things (but not exclusively), a key press, a mouse click, or a touch to a panel of an input apparatus of the medical imaging apparatus.

In a preferred embodiment, the connection of the planning unit with the at least one medical imaging apparatus includes an exchange of data by means of a medical data management. A standard for such a medical data management is, for example, the DICOM (Digital Imaging and Communications in Medicine) standard. A high degree of compatibility is provided via the widespread use of the DICOM standard since the interoperability between systems of different manufacturers is possible.

Within the scope of the present invention, a planning unit for planning at least one examination of an examination subject with at least one medical imaging apparatus and a medical planning system (including a planning unit and at least one medical imaging apparatus) are provided for planning at least one examination of an examination subject.

The planning unit and the medical planning system thereby comprise a processing unit, a storage unit and an output unit, and are designed to implement the following steps:

connect the planning unit with the at least one medical imaging apparatus by means of the processing unit,

detect at least one input parameter of a database of the planning unit and at least one input parameter of a database of a subset of at least one medical imaging apparatus by means of the processing unit,

filter the database of the planning unit and the database of the subset of the at least one medical imaging apparatus depending on at least one detected input parameter, by means of the processing unit, and

determine at least one input information that includes a selection from available medical imaging apparatuses for the examination of the examination subject, by means of the processing unit.

Furthermore, the present invention concerns a non-transitory, computer-readable storage medium encoded with programming instructions, the storage medium being loaded into a memory unit of a programmable controller or of a computer of a planning unit and/or of a medical planning system. All or various embodiments of the method according to the invention that are described above can be executed when the programming instructions are executed in the controller or control device of the planning unit and/or of the medical planning system. The programming instructions may require program units (for example libraries and auxiliary functions) in order to realize the corresponding embodiments of the method. The programming instructions can be in source code that must still be compiled and linked or that must only be interpreted, or an executable code that has only to be loaded into the corresponding computer for execution.

The computer-readable storage medium can be, for example a DVD, a magnetic tape or a USB stick, on which electronically readable control information is stored.

The advantages of the planning unit according to the invention, of the medical planning system according to the invention, of the computer program according to the invention and of the computer-readable storage medium according to the invention essentially correspond to the advantages of the method according to the invention that are described above. Features, advantages or alternative embodiments that are mentioned above are also similarly applicable to the other aspects of the invention, and vice versa.

### BRIEF DESCRIPTION OF THE DRAWINGS

FIG. 1 schematically illustrates a medical planning system according to the invention.

FIG. 2 is a flowchart of an embodiment of the method according to the invention.

### DESCRIPTION OF THE PREFERRED EMBODIMENTS

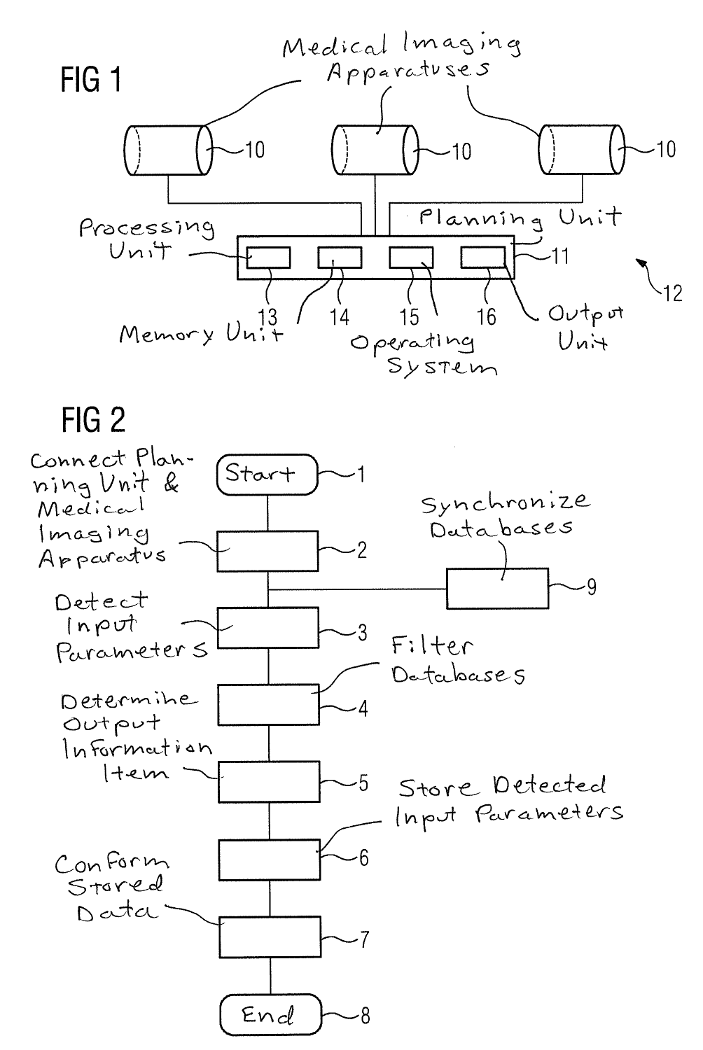

FIG. 1 shows a medical planning system 12 according to the invention, having a planning unit 11 and three medical imaging apparatuses 10.

The number of medical imaging apparatuses 10 that are used can deviate from the number indicated in this exemplary embodiment. The single requirement is the use of at least one medical imaging apparatus 10.

A planning unit 11 is a system that includes a processing unit 13, a memory unit 14, an operating system 15, and an output unit 16.

For example, the processing unit 13 or a processor (central processing unit, CPU) is a microprocessor or digital signal processor (DSP). The processor that is controlled by the program (which can be divided into a number of program modules) writes data into the memory, reads data from the memory and processes the data. For example, the processor can also be executed as an (application-specific) field programmable (logic) gate array (field programmable gate array, FPGA).

For example, the memory unit 14 can be a read-only memory (ROM) such as electrically erasable programmable read-only memory (EEPROM) or Flash EEPROM, read/write memory (random access memory, RAM) and disk storage such as hard drive storage. The memory unit 14 can be used to store a program (for example an operating system or an application program) and/or data (in particular image data, instruction data, configuration data, parameter data, protocol data and sequence data).

The operating system 15 includes computer programs, administers the system resources (hardware components) of a computer (for example memory units such as working memory and hard disks, input and output units such as interfaces) and provides application programs. The operating system 15 thus forms an interface between the system resources and the application programs.

For example, the output unit 16 can be a monitor to present options, commands, parameter data, sequence data and/or for graphical output of image data etc.

After connecting the planning unit 11 with the three medical imaging apparatuses 10, a detection of an input parameter of a database of the planning unit 11 and at least one input parameter of a database of a subset of the three medical imaging apparatuses 10 takes place via the processing unit 13, and a filtering of the database of the planning unit 11 and the database of a subset of the three medical imaging apparatuses 10 takes place via the processing unit 13, depending on at least one detected input parameter. After this, an output information that includes a selection from available medical apparatuses 10 for the examination of the examination subject is determined, likewise via the processing unit 13.

If the three medical imaging apparatuses 10 comprise two MRT apparatuses and a CT apparatus, for example, and if a patient ID and an examination workflow (for example) are detected after connecting the planning unit 11 with the three medical imaging apparatuses 10, after filtering the database of the planning unit 11 and the database of the subset of the three medical imaging apparatuses 10 depending on the detected input parameters the output information only includes the selection from the two MRT apparatuses. If the input parameter of the database of the subset of the three medical imaging apparatuses includes additional boundary conditions about an examination that are possible only at one of the two MRT apparatuses (for example specific measurement protocols), the output information thus only includes this one MRT apparatus.

FIG. 2 shows a flowchart of an embodiment of the method according to the invention. The method includes the method steps 1 through 9. In the following explanation, reference characters introduced in connection with FIG. 1 are used for the description of the method steps 1 through 9.

Method step 1 identifies the start of the planning of at least one examination of an examination subject with at least one medical imaging apparatus 10 by means of a planning unit 11.

In method step 2, the planning unit 11 is connected with the at least one medical imaging apparatus 10. The connection takes place via a network (for example internet or intranet); an authentication advantageously takes place via a user recognition and a password. The planning unit 11 can be connected wirelessly or via wires with the medical imaging apparatus 10. The connection can also take place with the assistance of a framework such as, for example, Active Server Pages.NET (ASP.NET).

The connection of the planning unit 11 with the at least one medical imaging unit 10 additionally includes an exchange of data by means of a medical data management. One standard for such a medical data management is, for example, the DICOM (Digital Imaging and Communications in Medicine) standard. For example, using the DICOM standard the input parameters of patient ID and job ID are input into a DICOM Modality Work List (DMWL) by means of the planning unit 11, which DICOM Modality Work List is then queried in turn at the medical imaging apparatus 10 that was designated for the examination of the examination subject. Data input with the aid of the planning unit 11 can be evaluated by means of the job ID. Selected measurement protocols or parameters of measurement protocols can be loaded into a controller of the measurement workflow to implement the examination, and the determined input parameters can be applied to elements of the controller of the measurement workflow.

In method step 9, after the connection of the planning unit 11 with the at least one medical imaging apparatus 10 a database of the planning unit 11 and a database of the at least one medical imaging apparatus 10 are synchronized with one another with regard to a uniform data inventory.

In method step 3, at least one input parameter of the database of the planning unit 11 and at least one input parameter of the database of a subset of the at least one medical imaging apparatus 10 are detected. This detection takes place manually via a user interface (for instance an input unit of the planning unit 11) or automatically using a pre-established routine (thus using pre-established detection criteria).

Method step 4 identifies a filtering of the database of the planning unit 11 and of the database of the at least one medical imaging apparatus 10 depending on at least one of the detected input parameters. This filtering advantageously takes place automatically depending on at least one of the detected input parameters. For example, a detected input parameter can represent a query; in this case, the filtering of the database according to the query criterion delivers the result of this query. Various detected input parameters can represent a combination of different query criteria, for example, and thus further limit the result of the query given an AND-linking or expand it accordingly given an OR-linking.

In method step 5, at least one item of output information that includes a selection from the available medical imaging apparatuses 10 for the examination of the examination subject is determined. This determination takes place depending on the filtered database, i.e. using the result of the query that was previously made, i.e. thus using the previously detected input parameters. The output information includes a subset of the complete set of medical imaging apparatuses that are available for an examination of the examination subject. A selection of a specific, preferred medical imaging apparatus can then take place manually via the user interface (for instance the input unit of the planning unit) or automatically using a pre-established routine (thus using pre-established detection criteria).

With method step 6, the at least one detected input parameter of the database of the planning unit 11 and/or the at least one detected input parameter of the database of the subset of the at least one medical imaging apparatus 10 and/or the selection from the available medical imaging apparatuses 10 are stored in the database of the planning unit 11 and/or in the database of the subset of the at least one medical imaging apparatus 10.

The at least one input parameter of the database of the planning unit 11 and/or the at least one input parameter of the database of the subset of the at least one medical imaging apparatus 10 can thereby also be input by means of the planning unit 11 and/or by means of the at least one medical imaging apparatus 10.

The at least one input parameter of the database of the planning unit 11 includes at least one parameter for identification of the examination subject, for instance an individual patient identification number (patient ID) and/or at least one parameter for identification of the examination, for instance an individual examination identification number (examination ID) or an identification number for an individual planning job (job ID).

The at least one input parameter of the database of the subset of the at least one medical imaging apparatus 10 includes at least one measurement protocol (for example a specific examination workflow); at least one parameter of a measurement protocol (for example a boundary condition of the specific examination workflow); at least one parameter of a hardware revision (for example a specific model of the medical imaging apparatus 10; and/or at least one parameter of a software revision (for example the revision number of an operating software of the medical imaging apparatus 10). Examination workflows are, for example, a cardiac MRT, a paranasal sinus CT etc.; boundary conditions of the specific examination workflow are, for example, an examination duration, a radiation intensity, a magnetic field strength etc. This type of input parameter allows a specific filtering of the databases with regard to suitable medical imaging apparatuses 10, meaning that only those medical imaging apparatuses 10 that are also considered for the concrete examination workflows, and satisfy all boundary conditions of the specific examination workflows as well as possible requirements for hardware and software of the medical imaging apparatuses, can be chosen for an examination.

At least one input parameter of the database of the planning unit 11 and/or at least one input parameter of the database of the subset of the at least one medical imaging apparatus 10 include an operator information, for example an information that is relevant to the examination of the examination subject, be it an instruction to the examination personnel, to an operator of the medical imaging apparatus 10 or to the maintenance personnel of the medical imaging apparatus 10, or even be it merely information about a medical imaging apparatus 10. For example, the operator information includes a continuous text, additional measurement parameters or other information that is relevant to the examination of the examination subject. Efficient examinations adapted to the operator information are thereby possible.

In method step 7, the stored data of the at least one medical imaging apparatus 10 are confirmed in the database of the planning unit 11 and/or in the database of the subset of the at least one medical imaging apparatus 10. This serves to avoid errors as they can arise in a rescheduling of an examination. For example, it is thus prevented that an examination cannot be implemented after changing a specified medical imaging apparatus 10 since a measurement protocol or a parameter of a measurement protocol is not available at the changed medical imaging apparatus 10. By confirming the stored data at the changed medical imaging apparatus 10, there the stored data can be reviewed again by a user and checked for consistency. The confirmation can thereby take place with a key press, a mouse click or touching a panel of an input device of the medical imaging apparatus 10.

Method step 8 identifies the end of the planning of at least one examination of an examination subject with at least one medical imaging apparatus 10 by means of a planning unit 11.

In summary, the invention concerns a method to plan at least one examination of an examination subject with at least one medical imaging apparatus by means of a planning unit, which includes the following steps:

connect the planning unit with the at least one medical imaging apparatus, detect at least one input parameter of a database of the planning unit and at least one input parameter of a database of a subset of at least one medical imaging apparatus, filter the database of the planning unit and the database of the subset of the at least one medical imaging apparatus depending on at least one detected input parameter, and determine at least one item of output information that includes a selection from medical imaging apparatuses available for the examination of the examination subject.

Although modifications and changes may be suggested by those skilled in the art, it is the intention of the inventors to embody within the patent warranted hereon all changes and modifications as reasonably and properly come within the scope of their contribution to the art.

We claim as our invention:

1. A method for planning at least one examination of an examination subject using at least one medical imaging apparatus comprising:
placing a computerized planning unit in communication with said at least one medical imaging apparatus;
detecting at least one input parameter of a database of the planning unit and at least one input parameter of a database of a subset of said at least one medical imaging apparatus;
filtering the database of the planning unit and the database of the subset of the at least one medical imaging apparatus depending on said at least one detected input parameter; and
in said planning unit, automatically determining at least one item of output information that includes a selection of a medical imaging apparatus that is available for examination of the examination subject, and emitting said at least one item of output information in electronic form at an output of said planning unit.

2. A method according to claim 1, wherein the at least one detected input parameter of the database of the planning unit and/or of the at least one detected input parameter of the database of the subset of the at least one medical imaging apparatus and/or the determination of the at least one item of output information that includes a selection from the available medical imaging apparatuses are stored in the database of the planning unit and/or in the database of the subset of the at least one medical imaging apparatus.

3. A method according to claim 2, wherein a parameter for identification of the examination is additionally associated with said examination and stored in the database of the planning unit and/or in the database of the subset of the at least one medical imaging apparatus.

4. A method according to claim 2, wherein after storing the at least one detected input parameter the database of the planning unit and/or of the at least one detected input parameter of the database of the subset of the at least one medical imaging apparatus and/or the selection from the available medical imaging apparatuses in the database of the planning unit and/or in the database of the subset of the at least one medical imaging apparatus, the stored data are confirmed by the at least one medical imaging apparatus.

5. A method according to claim 1, wherein the database of the planning unit and the database of the subset of the at least one medical imaging apparatus are synchronized with one another after placing the planning unit in communication with the at least one medical imaging apparatus.

6. A method according to claim 1, wherein the at least one input parameter of the database of the planning unit and/or the at least one input parameter of the database of the subset of the at least one medical imaging apparatus is entered via the planning unit and/or via the at least one medical imaging apparatus.

7. A method according to claim 1, wherein the at least one input parameter of the database of the planning unit includes at least one parameter for identification of the examination apparatus and/or at least one parameter for identification of the examination.

8. A method according to claim 1, wherein the at least one input parameter of the database of the subset of the at least one medical imaging apparatus includes at least one measurement protocol, at least one parameter of a measurement protocol, at least one parameter of a hardware revision and/or at least one parameter of a software revision.

9. A method according to claim 1, wherein at least one input parameter of the database of the planning unit and/or at least one input parameter of the database of the subset of the at least one medical imaging apparatus include an operator information.

10. A method according to claim 1, comprising, in said communication between the planning unit and the at least one medical imaging apparatus, exchanging of data via a medical data management.

11. A computerized planning unit for planning at least one examination of an examination subject with at least one medical imaging apparatus, said planning unit comprising:
an input that places a computerized processing unit in communication with said at least one medical imaging apparatus;
a database;
an operating unit configured to detect at least one input parameter of the database and at least one input parameter of a database of a subset of said at least one medical imaging apparatus;
said operating unit being configured to filter the database and the database of the subset of the at least one medical imaging apparatus depending on said at least one detected input parameter;
said operating unit being configured to automatically determine at least one item of output information that includes a selection of a medical imaging apparatus that is available for examination of the examination subject; and
an output unit that emits said at least one item of output information in electronic form.

12. A medical planning system comprising:
a computerized planning unit;
at least one medical imaging apparatus in communication with said planning unit;
said planning unit being configured to detect at least one input parameter of a database of the planning unit and at least one input parameter of a database of a subset of said at least one medical imaging apparatus;
said planning unit being configured to filter the database of the planning unit and the database of the subset of the at least one medical imaging apparatus depending on said at least one detected input parameter; and
said planning unit being configured to automatically determine at least one item of output information that includes a selection of a medical imaging apparatus that is available for examination of the examination subject, and emit said at least one item of output information in electronic form at an output of said planning unit.

13. A non-transitory, computer-readable data storage medium encoded with programming instructions, said storage medium being loaded into a computerized planning unit, and said programming instructions causing said computerized planning unit to:
Place the computerized planning unit in communication with at least one medical imaging apparatus;
detect at least one input parameter of a database of the planning unit and at least one input parameter of a database of a subset of said at least one medical imaging apparatus;
filter the database of the planning unit and the database of the subset of the at least one medical imaging apparatus depending on said at least one detected input parameter; and
automatically determine at least one item of output information that includes a selection of a medical imaging apparatus that is available for examination of the examination subject, and emit said at least one item of output information in electronic form at an output of said planning unit.
